(19) United States
(12) Patent Application Publication
(10) Pub. No.: US 2009/0062657 A1
(43) Pub. Date: Mar. 5, 2009

(54) ULTRASONIC DIAGNOSTIC APPARATUS

(76) Inventors: Koji Yanagihara, Tokyo (JP); Kiyoshi Matsumura, Tokyo (JP); Osamu Furuta, Tokyo (JP); Akimasa Asai, Tokyo (JP); Kazuhiko Hayakawa, Tokyo (JP); Hiroyuki Hatakenaka, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/200,852

(22) Filed: Aug. 28, 2008

(30) Foreign Application Priority Data
Aug. 31, 2007 (JP) 2007-226055
May 27, 2008 (JP) 2008-137509

(51) Int. Cl. A61B 8/00

(52) U.S. Cl. 600/459

(57) ABSTRACT

An ultrasonic diagnostic apparatus includes an operating device for an operator to input an instruction, a probe for transmitting and receiving ultrasonic waves, an image display device for displaying an ultrasonic image, and a processing device. The processing device controls driving the probe based on the instruction, generates the ultrasonic image based on a received signal, and displays the ultrasonic image on the image display device. The operating device is formed separately from the processing device and the image display device is supported by an arm extending from a column support along the processing device or by an arm extending from the processing device.

### CROSS REFERENCE TO RELATED APPLICATIONS

This application claims the benefit of Japanese Patent Application No. 2007-226055 filed Aug. 31, 2007, and Japanese Patent Application No. 2008-137509 filed May 27, 2008, both of which are herein incorporated by reference in their entirety.

### BACKGROUND OF THE INVENTION

The subject matter disclosed herein relates to an ultrasonic diagnostic apparatus, and in particular, relates to an ultrasonic diagnosis apparatus that enables effective use of a space of a room in which the ultrasonic diagnostic apparatus is installed.

A conventional ultrasonic diagnostic apparatus includes: an operating device for an operator to input an instruction; a probe for transmitting and receiving ultrasonic waves; an image display device for displaying an ultrasonic image; and a processing device which controls driving the probe, generating the ultrasonic image based on a received signal, and displaying it. The operating device, probe, image display device, and processing device can move as one unit (for example, see Japanese Unexamined Patent Publication No. 2003-339708.)

In order to locate an operating device within the reach of an operator, a conventional ultrasonic diagnostic apparatus is placed on the side of the operator with respect to a bed on which a patient lies.

However, devices from the operating device through the processing device are formed as one unit. Therefore, when the large conventional ultrasonic diagnostic apparatus is placed on the side of the operator with respect to the bed, a considerable part of a space for the operator is occupied.

### BRIEF DESCRIPTION OF THE INVENTION

It is desirable that the problem described previously is solved.

In a first aspect of the invention, there is provided an ultrasonic diagnostic apparatus, including: an operating device (1) for an operator to input an instruction; a probe (2) for transmitting and receiving ultrasonic waves; an image display device (3) for displaying an ultrasonic image; and an processing device (4) which controls driving the probe (1) based on the instruction, generating the ultrasonic image based on a received signal, and displaying it on the image display device (3), wherein the operating device (1) is formed separately from the processing device (4); and wherein the processing device (4) is mounted on a mounting platform (5) whose portion being equal to or lower than 70 cm from a floor (F) is 10 cm thick or less or the processing device (4) is placed on the floor (F) and its portion being equal to or lower than 70 cm from the floor (F) is 10 cm thick or less.

According to the ultrasonic diagnostic apparatus of the first aspect, the operating device (1) and the processing device (4) are formed separately. Therefore, it is possible to place the operating device (1) on the side of the operator with respect to a bed (B) on which a patient lies, and to place the processing device (4) on the other side of the bed (B). Further, when the processing device (4) is mounted on the mounting platform (5) whose portion being equal to or less than 70 cm from the floor (F) is 10 cm thick or less, or the processing device (4) is placed on the floor (F), the thickness of the portion of the processing device (4) being equal to or less than 70 cm from the floor (F) is 10 cm or less. As a result, suppose a spacing between the bed (B) and the wall (W) is 11 cm, for example, the processing device (4) can be installed along the wall (W). Further, a height of an ordinary bed (B) is less than 70 cm. As a result of the above, the space for the operator becomes larger than a conventional space, making it possible to effectively utilize the space of the room in which the ultrasonic diagnostic apparatus is installed.

In a second aspect of the invention, there is provided an ultrasonic diagnostic apparatus according to the first aspect, wherein a back surface of the processing device (4) or the mounting platform (5) is not uneven or a level of unevenness is 1 cm or less.

According to the ultrasonic diagnostic apparatus of the second aspect, the back surface of the processing device (4) or the mounting platform (5) is substantially flat. Therefore, with almost no spacing provided, the processing device (4) can be installed along the wall (W).

In a third aspect of the invention, there is provided an ultrasonic diagnostic apparatus according to the first or second aspect, wherein the processing device (4) or the mounting platform (5) has a tip-resistant leg (16).

According to the ultrasonic diagnostic apparatus of the third aspect, even if a length of a contact surface in the thickness direction of the processing device (4) or the mounting platform (5) is 10 cm or less, the processing device (4) or the mounting platform (5) is prevented from tipping over by the tip-resistant leg (16).

In a fourth aspect of the invention, there is provided an ultrasonic diagnostic apparatus according to any one of the first to third aspects, wherein the image display device (3) is supported by an arm (17) extending from a column support (19) provided along the processing device (4) or by an arm (17) extending from the processing device (4).

According to the ultrasonic diagnostic apparatus of the fourth aspect, the image display device (3) is supported by the arm (17) extending from the column support (19) along the processing device (4) or by the arm (17) extending from the processing device (4). Therefore, the space on the side of the operator with respect to the bed (B) is not occupied for supporting the image display device (3). As a result, the space for the operator becomes larger than the conventional space. Accordingly, the space in the room in which the ultrasonic diagnostic apparatus is installed can effectively be utilized.

In a fifth aspect of the invention, there is provided an ultrasonic diagnostic apparatus according to the fourth aspect, wherein the arm (17) can rotate or expand and contract.

According to the ultrasonic diagnostic apparatus of the fifth aspect, the arm (17) can rotate or expand and contract. As a result, a spatial position or an orientation of the image display device can be changed.

In a sixth aspect of the invention, there is provided an ultrasonic diagnostic apparatus according to any one of the first to fifth aspects, wherein the operating device (1) is mounted on a movable platform (18) with a caster.

According to the ultrasonic diagnostic apparatus of the sixth aspect, the position of the operating device (1) can freely be changed by moving the movable platform (18).

In a seventh aspect of the invention, there is provided an ultrasonic diagnostic apparatus, including: an operating device (1) for an operator to input an instruction; a probe (2) for transmitting and receiving ultrasonic waves; an image display device (3) for displaying an ultrasonic image; and a processing device (4) which controls driving the probe (1) based on the instruction, generating the ultrasonic image based on a received signal, and displaying it on the image display device (3), wherein the operating device (1) is formed separately from the processing device (4); and wherein the image display device (3) is supported by an arm (17) extending from a column support (19) along the processing device (4) or by an arm (17) extending from the processing device (4).

According to the ultrasonic diagnostic apparatus of the seventh aspect, the operating device (I) and the processing device (4) are formed separately. Therefore, the operating device (1) can be placed on the side of the operator with respect to a bed (B) on which a patient lies and the processing device (4) can be placed on the other side of the bed (B). Further, the image display device (3) is supported by the arm (17) extending from the column support (19) along the processing device (4) or by the arm (17) extending from the processing device (4). Therefore, the space on the side of the operator with respect to the bed (B) is not occupied for supporting the image display device (3). As a result, the space for the operator becomes larger than the conventional space. Accordingly, the space in the room in which the ultrasonic diagnostic apparatus is installed can effectively be utilized.

In an eighth aspect of the invention, there is provided an ultrasonic diagnostic apparatus according to the seventh aspect, wherein the arm (17) includes: a horizontal arm extending from the column support (19) or the processing device (4); and an up-and-down arm extending from the horizontal arm and supporting the image display device (3).

According to the ultrasonic diagnostic apparatus of the eighth aspect, horizontal and vertical positions can be adjusted by the horizontal arm and the up-and-down arm.

In a ninth aspect of the invention, there is provided an ultrasonic diagnostic apparatus according to the eighth aspect, wherein the horizontal arm rotates at a joint with the column support (19) or the processing device (4).

According to the ultrasonic diagnostic apparatus of the ninth aspect, the horizontal position can be changed as the horizontal arm rotates.

In a tenth aspect of the invention, there is provided an ultrasonic diagnostic apparatus according to the eighth or ninth aspect, wherein the horizontal arm includes: a first horizontal arm extending from the column support (19) or the processing device (4); and a second horizontal arm extending from the first horizontal arm. The second horizontal arm rotates at a joint with the first horizontal arm.

According to the ultrasonic diagnostic apparatus of the tenth aspect, the horizontal position can be changed as the first horizontal arm and the second horizontal arm rotate.

In an eleventh aspect of the invention, there is provided an ultrasonic diagnostic apparatus according to any one of the eighth to tenth aspects, wherein a length of the up-and-down arm is variable.

According to the ultrasonic diagnostic apparatus of the eleventh aspect, the vertical position can be changed by varying the length of the up-and-down arm.

In a twelfth aspect of the invention, there is provided an ultrasonic diagnostic apparatus according to any one of the eighth to eleventh aspects, wherein the up-and-down arm rotates at a joint with the horizontal arm in a horizontal direction. The rotation allows the image display device to rotate in a horizontal direction. Further, the image display device rotates vertically with respect to the up-and-down arm at a joint with the up-and-down arm.

According to the ultrasonic diagnostic apparatus of the twelfth aspect, the orientation of the image display device can be changed.

In a thirteenth aspect of the invention, there is provided an ultrasonic diagnostic apparatus according to any one of the eighth to twelfth aspects, wherein the operating device (1) is mounted on a movable platform (18) with a caster.

According to the ultrasonic diagnostic apparatus of the thirteenth aspect, the position of the operating device (1) can freely be changed by moving the movable platform (18).

In a fourteenth aspect of the present invention, there is provided an ultrasonic diagnostic apparatus according to the seventh aspect, wherein the arm (17) includes: a first parallel link arm (17d) extending from the column support (19) or the processing device (4) in a rotatable and vertically deformable manner; a second parallel link arm (17e) extending from the first parallel link arm (17d) in a vertically deformable manner; and a rotating shaft (17f) extending from the second parallel link arm (17e) and rotatably supporting the image display device (3).

According to the ultrasonic diagnostic apparatus of the fourteenth aspect, horizontal and vertical positions can be adjusted by use of the first parallel link arm, the second parallel link arm, and the rotating shaft.

In a fifteenth aspect of the present invention, there is provided an ultrasonic diagnostic apparatus according to the seventh aspect, wherein the arm (17) includes: a first horizontal arm (17a) extending from the column support (19) or the processing device (4) in a rotatable manner; a second parallel link arm (17e) extending from the first horizontal arm (17a) in a vertically deformable manner; and a rotating shaft (17f) extending from the second parallel link arm (17e) and rotatably supporting the image display device (3).

According to the ultrasonic diagnostic apparatus of the fifteenth aspect, horizontal and vertical positions can be adjusted by use of the first horizontal arm, the second parallel link arm, and the rotating shaft.

In a sixteenth aspect of the present invention, there is provided an ultrasonic diagnostic apparatus according to the seventh aspect, wherein the arm (17) comprises: a first slide guide (17g) extending from the column support (19) or the processing device (4); a second slide guide (17h) supported slidably along the first slide guide (17g) and extending in a direction crossing a sliding direction; and an up-and-down arm (17c) supported slidably along the second slide guide (17h) and supporting the image display device (3) in a vertically movable and rotatable manner.

According to the ultrasonic diagnostic apparatus of the sixteenth aspect, horizontal and vertical positions can be adjusted by use of the first slide guide, the second slide guide, and the up-and-down arm.

In a seventeenth aspect of the present invention, there is provided an ultrasonic diagnostic apparatus according to the seventh aspect, wherein the arm (17) includes: a second slide guide (17h) extending from the column support (19) or the processing device (4) in a rotatable manner; and an up-and-down arm (17c) supported slidably along the second slide guide (17h) and supporting the image display device (3) in a vertically movable and rotatable manner.

According to the ultrasonic diagnostic apparatus of the seventeenth aspect, horizontal and vertical positions can be adjusted by use of the second slide guide and the up-and-down arm.

In an eighteenth aspect of the present invention, there is provided an ultrasonic diagnostic apparatus according to the seventh aspect, wherein the arm (17) includes: an up-and-down rotating pole (17i) installed in the column support (19) or the processing device (4); a second slide guide (17h) extending from the up-and-down rotating pole (17i); and a rotating shaft (17f) supported slidably along the second slide guide (17h) and rotatably supporting the image display device (3).

According to the ultrasonic diagnostic apparatus of the eighteenth aspect, horizontal and vertical positions can be adjusted by use of the up-and-down rotating pole, second slide guide, and the rotating shaft.

In a nineteenth aspect of the present invention, there is provided an ultrasonic diagnostic apparatus according to any one of the first to eighteenth aspects, wherein the operating device (1) and the processing device (4) have radio communication means (1a) and (4a), respectively, for mutual radio communication.

According to the ultrasonic diagnostic apparatus of the nineteenth aspect, a wire for connecting the operating device (1) with the processing device (4) can be eliminated. Therefore, the operating device (1) can be moved freely.

In a twentieth aspect of the present invention, there is provided an ultrasonic diagnostic apparatus according to any one of the first to nineteenth aspects, wherein a cable receiving space (5a) is provided inside the processing device (4) or the mounting platform (5) for receiving a cable (6) of the probe (2).

According to the ultrasonic diagnostic apparatus of the twentieth aspect, the cable (6) of the probe (2) can be received inside the processing device (4) or the mounting platform (5). Therefore, the cable (6) is prevented from being in the way.

In a twenty-first aspect of the present invention, there is provided an ultrasonic diagnostic apparatus according to the twentieth aspect, wherein an opening (5b) is provided at a position equal to or higher than 120 cm from the floor (F) of the processing device (4) or the mounting platform (5); and wherein the cable (6) of the probe (2) is received in the cable receiving space (5a) through the opening (5b).

According to the ultrasonic diagnostic apparatus of the twenty-first aspect, the opening (5b) of the cable receiving space (5a) is provided at the position equal to or higher than 120 cm from the floor (F). Therefore, the cable (6) comes in and goes out at a position sufficiently higher than that of the patient lying on the bed (B), which prevents the cable (6) from giving the patient discomfort.

In a twenty-second aspect of the present invention, there is provided an ultrasonic diagnostic apparatus according to the twentieth or twenty-first aspect, wherein a cable stopper (7) is provided for preventing the cable (6) from coming into and going out of the cable receiving space (5a).

According to the ultrasonic diagnostic apparatus of the twenty-second aspect, the cable (6) is prevented from going out of the opening (5b) or being pulled into the cable receiving space (5a) against the operator's will.

In a twenty-third aspect of the present invention, there is provided an ultrasonic diagnostic apparatus according to the twenty-second aspect, wherein the cable stopper (7) is a slit (7a) which is narrower than an outside diameter of the cable (6).

According to the ultrasonic diagnostic apparatus of the twenty-third aspect, the cable (6) is inserted in the slit (7a) so that the cable (6) is stopped in an engaged state by the elasticity of the cable (6), and the structure can also be simple.

In a twenty-fourth aspect of the present invention, there is provided an ultrasonic diagnostic apparatus according to any one of the twenty-first to twenty-third aspects, wherein there is provided, in the opening (5b), a roller (8) which can contact an undersurface of the cable (6).

According to the ultrasonic diagnostic apparatus of the twenty-fourth aspect, the cable (6) comes into and goes out of the opening (5b) smoothly because of the roller (8).

In a twenty-fifth aspect of the present invention, there is provided an ultrasonic diagnostic apparatus according to any one of the twenty-first to twenty-fourth aspects, wherein a probe connector (9) is provided above the opening (5b) of the processing device (4).

According to the ultrasonic diagnostic apparatus of the twenty-fifth aspect, the probe connector (9) is provided above the opening (5b). Therefore, the cable connector (6a) of the cable (6) can be attached to or detached from the probe connector (9) of the processing device (4) without being disturbed by the cable (6).

In a twenty-sixth aspect of the present invention, there is provided an ultrasonic diagnostic apparatus according to the twenty-fifth aspect, wherein the cable (6) is bent in the shape of a U from the opening (5b), to be received into the cable receiving space (5a).

According to the ultrasonic diagnostic apparatus of the twenty-sixth aspect, only one portion at the lower end of the U-shaped cable (6) is bent with a small curvature, preventing the cable (6) from being damaged.

In a twenty-seventh aspect of the present invention, there is provided an ultrasonic diagnostic apparatus according to the twenty-sixth aspect, wherein a movable pulley (10) is hung from a lower end of the U-shaped cable (6).

According to the ultrasonic diagnostic apparatus of the twenty-seventh aspect, the cable (6) is prevented from being bent with a curvature smaller than a radius of the movable pulley (10).

In a twenty-eighth aspect of the present invention, there is provided an ultrasonic diagnostic apparatus according to the twenty-seventh aspect, wherein the movable pulley (10) is urged downwards by a weight (11).

According to the ultrasonic diagnostic apparatus of the twenty-eighth aspect, the weight (11) provides a force to smoothly pull the cable (6) into the cable receiving space (5a). In a twenty-ninth aspect of the present invention, there is provided an ultrasonic diagnostic apparatus according to the twenty-seventh or twenty-eighth aspect, wherein the movable pulley (10) is supported on a rail (12) in a vertically movable manner.

According to the ultrasonic diagnostic apparatus of the twenty-ninth aspect, since the movable pulley (10) vertically moves while being supported on the rail (12), its stability and reliability are improved.

In a thirtieth aspect of the present invention, there is provided an ultrasonic diagnostic apparatus according to any one of the twenty-seventh to twenty-ninth aspects, wherein a cable lock (13) is installed to prevent the disconnection of the cable (6) from the movable pulley (10).

According to the ultrasonic diagnostic apparatus of the thirtieth aspect, the disconnection of the cable (6) from the movable pulley (10) is prevented, improving the stability and reliability.

In a thirty-first aspect of the present invention, there is provided an ultrasonic diagnostic apparatus according to the thirtieth aspect, wherein the cable lock (13) consists of a rotatable lever-like member.

According to the ultrasonic diagnostic apparatus of the thirty-first aspect, the disconnection of the cable (6) from the movable pulley (10) is prevented by a simple structure.

In a thirty-second aspect of the present invention, there is provided an ultrasonic diagnostic apparatus according to the thirtieth or thirty-first aspect, wherein a cover (14) is provided over a portion of the processing device (4) or the mounting platform (5) 120 cm or less from the floor (F) which slides vertically to allow the access to the inside.

According to the ultrasonic diagnostic apparatus of the thirty-second aspect, the cover (14) is vertically slid to allow the access to an inner portion of the processing device (4) or the mounting device (5) 120 cm or less from the floor (F), making the maintenance work easier.

In a thirty-third aspect of the present invention, there is provided an ultrasonic diagnostic apparatus according to the thirty-second aspect, wherein the cable (6) can be attached to or detached from the movable pulley (10) when the cable lock (13) is released.

According to the ultrasonic diagnostic apparatus of the thirty-third aspect, when removing a probe (2) not to be used from the processing device (4), it becomes possible to remove the cable (6) from the movable pulley (10) and, also, when attaching a probe (2) to be used to the processing device (4), it becomes possible to mount the cable (6) on the movable pulley (10).

In a thirty-fourth aspect of the present invention, there is provided an ultrasonic diagnostic apparatus according to the thirty-second or twenty-third aspect, wherein a movable-pulley lock (15) is provided for holding the movable pulley (10) at a position of the rail (12) equal to or higher than 60 cm from the floor.

According to the ultrasonic diagnostic apparatus of the thirty-fourth aspect, the movable pulley (10) from which the cable (6) is removed can be held at the position equal to or higher than 60 cm from the floor, making it easy to mount a cable (6) of the probe (2) to be used next for the movable pulley (10).

In a thirty-fifth aspect of the present invention, there is provided an ultrasonic diagnostic apparatus according to the twenty-fourth aspect, wherein the movable-pulley lock (15) consists of a seesaw-like member that varies its position in a seesaw-like manner.

According to the ultrasonic diagnostic apparatus of the thirty-fifth aspect, the movable pulley (10) can be prevented from descending by a simple structure.

According to the ultrasonic diagnostic apparatus of the invention, the processing device (1) can be placed on the side of the operator with respect to the bed (B) on which the patient lies and the processing device (4) can be placed on the other side of the bed (B). As a result of the above, the space for the operator becomes larger than the conventional space, making it possible to effectively utilize the space in the room in which the ultrasonic diagnostic apparatus is installed.

The ultrasonic diagnostic apparatus of the invention can be used as a stationary ultrasonic diagnostic apparatus.

Further objects and advantages of the present invention will be apparent from the following description of the preferred embodiments of the invention as illustrated in the accompanying drawings.

### BRIEF DESCRIPTION OF THE DRAWINGS

FIG. 1 is a partially broken side view showing an ultrasonic diagnostic apparatus according to Embodiment 1.

FIG. 2 is a front view showing the ultrasonic diagnostic apparatus according to Embodiment 1.

FIG. 3 is a plan view showing the ultrasonic diagnostic apparatus according to Embodiment 1.

FIG. 4 is a partially broken enlarged side view of the vicinity of the opening.

FIG. 5 is a partially broken enlarged front view of the vicinity of the opening.

FIG. 6 is an enlarged side view of the vicinity of the movable pulley.

FIG. 7 is an enlarged side view of the vicinity of the movable-pulley lock (a state where the movable-pulley lock is released).

FIG. 8 is an enlarged side view of the vicinity of the movable-pulley lock (a state where the movable-pulley lock is working).

FIG. 9 is an enlarged side view showing a state where the cable lock is released.

FIG. 10 is a front view showing an ultrasonic diagnostic apparatus according to Embodiment 2.

FIG. 11 is a front view showing an ultrasonic diagnostic apparatus according to Embodiment 3.

FIG. 12 is a front view showing an ultrasonic diagnostic apparatus according to Embodiment 4.

FIG. 13 is a front view showing an ultrasonic diagnostic apparatus according to Embodiment 5.

FIG. 14 is an upward view showing a second slide guide according to Embodiment 5.

FIG. 15 is a front view showing an ultrasonic diagnostic apparatus according to Embodiment 6.

FIG. 16 is a front view showing an ultrasonic diagnostic apparatus according to Embodiment 7.

### DETAILED DESCRIPTION OF THE INVENTION

Hereinbelow, preferred embodiments of the invention will be described with reference to drawings. The embodiments are not to limit the invention thereto.

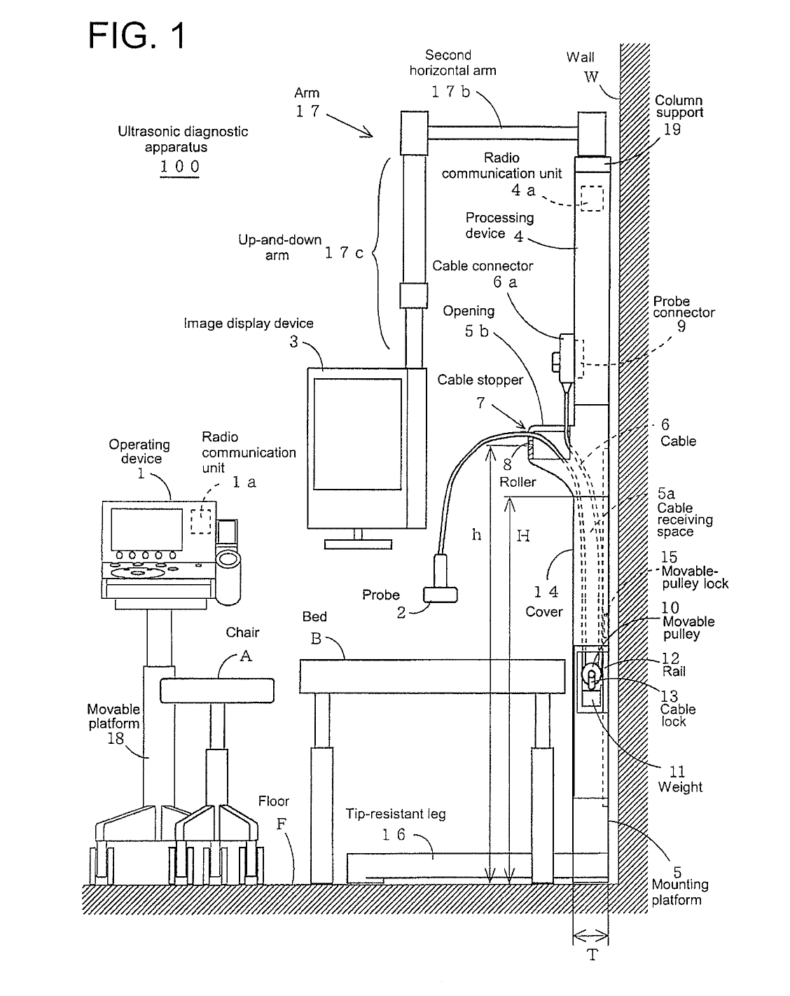

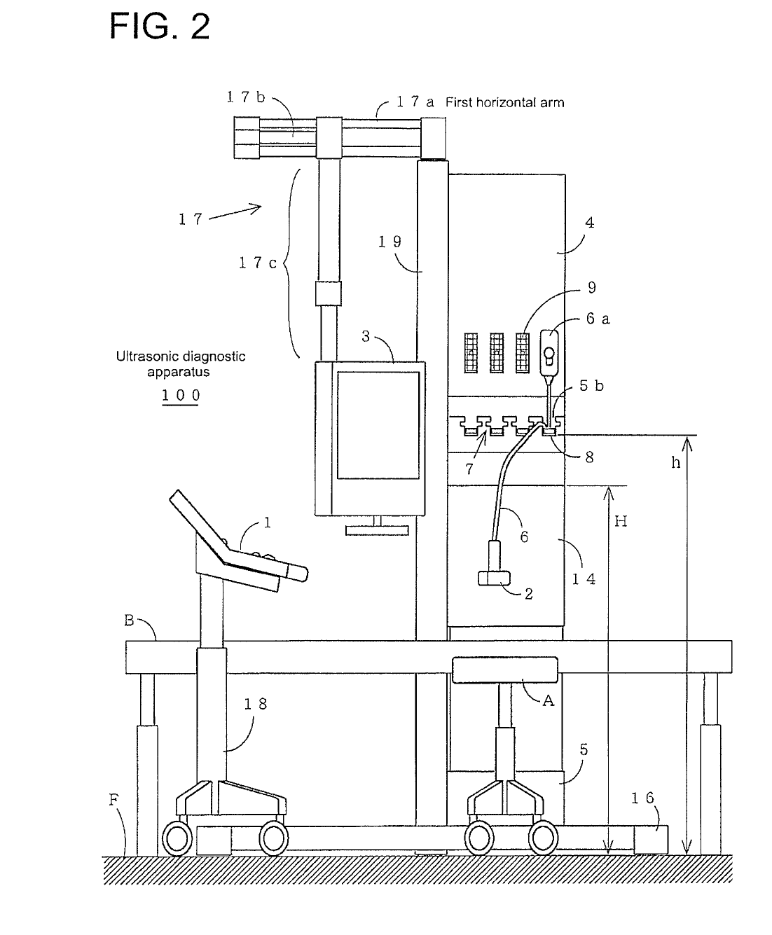

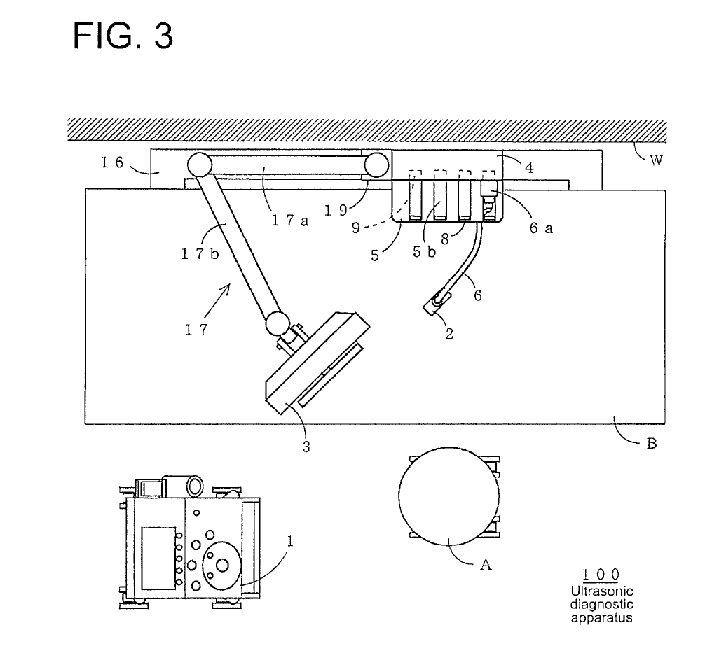

FIG. 1 is a partially broken side view showing an ultrasonic diagnostic apparatus 100 according to Embodiment 1. FIG. 2 is a front view showing the ultrasonic diagnostic apparatus 100. FIG. 3 is a plan view showing the ultrasonic diagnostic apparatus 100.

The ultrasonic diagnostic apparatus 100 basically includes: an operating device 1 for an operator to input an instruction; a probe 2 for transmitting and receiving ultrasonic waves; an image display device 3 for displaying an ultrasonic image; and a processing device 4 which controls driving the probe 1 based on the instruction by the operator, generating the ultrasonic image based on a received signal, and displaying it on the image display device 3.

The operating device 1 and the processing device 4 are formed separately. Further, the operating device 1 is installed on a movable platform 18 with a caster, and is placed on the side where a chair A on which the operator sits is placed with respect to a bed B.

Also, the operating device 1 has a radio communication unit 1a for radio communication with the processing device 4.

The processing device 4 is fixed to a mounting platform 5 placed between the bed B and a wall W.

Further, the processing device 4 has a radio communication unit 4a for radio communication with the operating device 1 and a probe connector 9 for connecting a cable connector 6a of the probe 2.

Back surfaces of the processing device 4 and the mounting platform 5 are not uneven or their levels of unevenness are approximately 1 cm or less, being substantially flat.

The height of the mounting platform 5 is approximately 150 cm.

The thickness T of a portion of the mounting platform 5 up to a height H of approximately 100 cm is approximately 10 cm. Further, a portion of the mounting platform 5 between approximately 100 cm and approximately 130 cm in height is a projection, whose thickness is approximately 20 cm.

An inner portion of the mounting platform 5 serves as a cable receiving space 5a for receiving a cable 6 of the probe 2. When a cover 14 of the mounting platform 5 is slid downwards, the inner portion of the mounting platform 5 between approximately 65 cm and approximately 100 cm in height (H) is exposed, allowing the operator to access the cable receiving space 5a.

Further, the mounting platform 5 has a tip-resistant leg 16. The tip-resistant leg 16 is preferably fastened to a floor F with anchor bolts.

The image display device 3 is supported by an arm 17 extending from a column support 19 fixed to side faces of the processing device 4 and the mounting platform 5.

The arm 17 includes: a first horizontal arm 17a extending horizontally from the column support 19; a second arm 17b extending horizontally from the first horizontal arm 17a; and an up-and-down arm 17c extending vertically from the second arm 17b.

The first horizontal arm 17a rotates horizontally at a joint with the column support 19. Further, the second horizontal arm 17b rotates horizontally at a joint with the first horizontal arm 17a.

The length of the up-and-down arm 17c is variable. Also, the up-and-down arm 17c rotates horizontally at a joint with the second horizontal arm 17b. The lower end of the up-and-down arm 17c serves as a joint that allows the image display device 3 to rotate within a vertical plane.

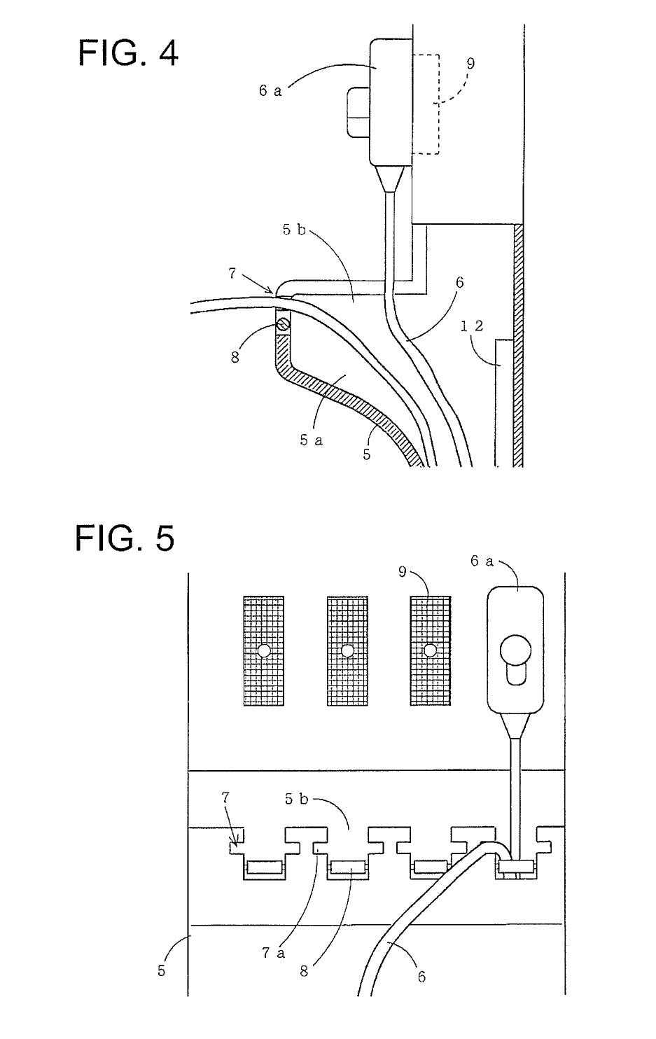

As shown in FIGS. 4 and 5, the projected portion of the mounting platform 5 between approximately 120 cm and approximately 130 cm in height (h) serves as an opening 5b.

The cable 6 of the probe 2 is bent in the shape of a U and is received in the cable receiving space 5a through the opening 5b.

A cable stopper 7 is formed in the opening 5b.

The cable stopper 7 is a slit 7a that is narrower than an outside diameter of the cable 6. By inserting the cable 6 in the slit 7a, the cable 6 can be stopped in an engaged state by the elasticity of the cable 6.

Moreover, in the opening 5b, there is provided a roller 8 which can contact an undersurface of the cable 6.

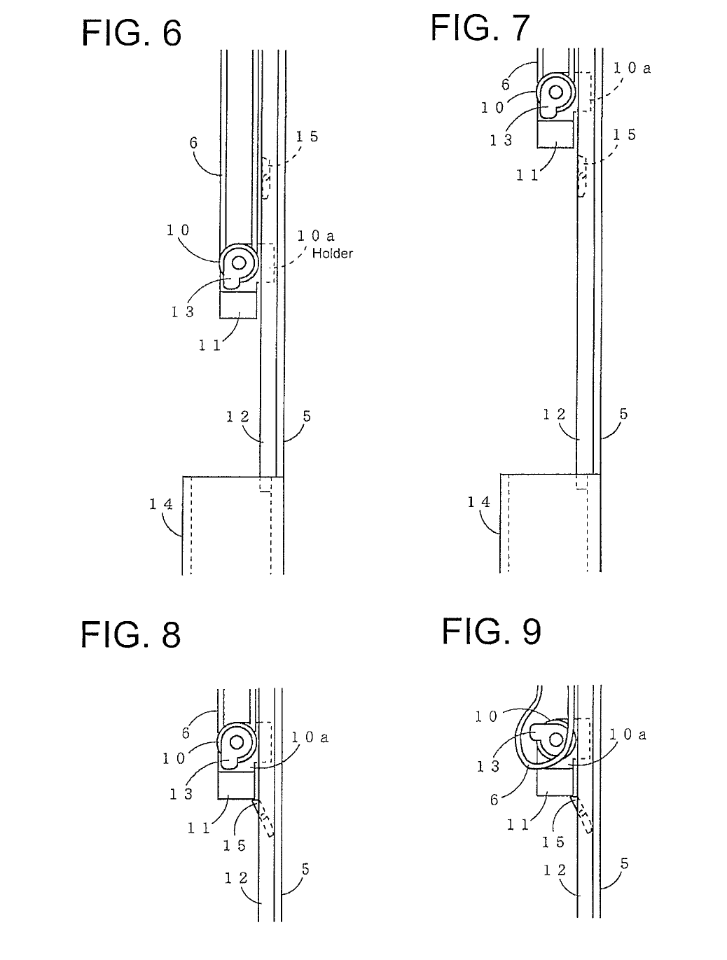

FIG. 6 shows a state in which the cover 14 is slid downwards and the inner portion of the mounting platform 5 between approximately 65 cm and approximately 100 cm in height (H) is exposed.

A movable pulley 10 whose diameter is approximately 10 mm or larger, for example, is hung from a lower end of the U-shaped cable 6.

The movable pulley 10 is supported in a vertically movable manner, by a holder 10a, on a rail 12 fixed at right angles to the back surface of the mounting platform 5.

Also, the movable pulley 10 is urged downwards by a weight 11.

A cable lock 13 is installed in the movable pulley 10.

The cable lock 13 includes a rotatable lever-like member.

A movable-pulley lock 15 is installed at a position of approximately 70 cm in height of the rail 12 from the floor F.

The movable-pulley lock 15 is a seesaw-like member that moves in a seesaw-like manner and changes its position.

FIGS. 7 to 9 show steps to release the cable 6 from the movable pulley 10.

First, the cover 14 is slid downwards. In a state where the inner portion of the mounting platform 5 between approximately 65 cm and approximately 100 cm in height (H) is exposed, the cable 6 is pulled out. Further, as shown in FIG. 7, the movable pulley 10 is raised to a position above the movable-pulley lock 15.

Then, as shown in FIG. 8, the movable-pulley lock 15 is made to project, the movable pulley 15 is lowered, and the movable pulley 15 is mounted on the movable-pulley lock 15.

Next, as shown in FIG. 9, the cable lock 13 is rotated and raised by fingers to release the cable 6.

Steps to hang the movable pulley 10 from the cable 6 are carried out in a reversed manner.

According to the ultrasonic diagnostic apparatus 100 of Embodiment 1, the following advantages are obtained.

(a) The operating device 1 and the processing device 4 are formed separately. Therefore, it is possible to place the operating device 1 on the side of the operator with respect to the bed B on which the patient lies and to place the processing device 4 on the other side of the bed B. Also, the processing device 4 can be installed along the wall W between the bed B and the wall W. As a result of the above, the space for the operator becomes larger than a conventional space and it becomes possible to effectively utilize the space of the room in which the ultrasonic diagnostic apparatus 100 is installed.

(b) The back surface of the processing device 4 or the mounting platform 5 is substantially flat. Therefore, the processing device 4 can be installed along the wall W with almost no spacing.

(c) Although the length of the contact surface of the mounting platform 5 in its thickness direction is equal to 10 cm or less, the mounting platform 5 is prevented from tipping over by the tip-resistant leg 16.

(d) By moving the movable platform 18, the position of the operating device 1 can easily be changed.

(e) Since the wire for connecting the operating device 1 with the processing device 4 can be eliminated, the processing device 1 can freely be moved.

(f) The image display device 3 is supported by the arm 17 extending from the column support 19 provided along the processing device 4. As a result, the space on the side of the operator with respect to the bed B is not occupied for supporting the image display device 3. Thus, the space for the operator becomes larger than the conventional space, making it possible to effectively utilize the space of the room in which the ultrasonic diagnostic apparatus is installed.

(g) By rotating, expanding or contracting the arm 17, the spatial position or orientation of the image display device 3 can be changed.

(h) It is possible to receive the cable 6 of the probe 2 inside the mounting platform 5. Therefore, the cable 6 is prevented from being in the way.

(i) There is provided the opening 5b of the cable receiving space 5a at a position equal to 120 cm or higher from the floor F. Thus, the cable 6 comes in and goes out at a position sufficiently higher than that of the patient lying on the bed B, preventing the cable 6 from contacting and giving the patient discomfort.

(j) There is provided the cable stopper 7, which prevents the cable 6 from going out of the opening 5b or being pulled into the cable receiving space 5a against the operator's will.

(k) By inserting the cable 6 in the slit 7a, the cable 6 is stopped in an engaged state by the elasticity of the cable 6.

(l) The cable 6 can smoothly comes in and goes out of the opening 5b because of the roller 8.

(m) The probe connector 9 is provided above the opening 5b. Therefore, the cable connector 6a of the cable 6 can be attached to or detached from the probe connector 9 without being disturbed by the cable 6.

(n) The cable 6 is bent in the shape of a U to be received into the cable receiving space 5a from the opening 5b. Accordingly, only one portion at the lower end of the U-shaped cable 6 is bent, preventing the cable 6 from being damaged.

(o) The movable pulley 10 prevents the cable 6 from bending with the curvature smaller than the radius (for example, 5 mm or over) of the movable pulley 10.

(p) The weight 11 attached to the movable pulley 10 provides a force to smoothly pull the cable 6 into the cable receiving space 5a.

(q) Since the movable pulley 10 vertically moves while being supported on the rail 12, its stability and reliability is improved.

(r) Since the cable lock 13 is installed, the disconnection of the cable 6 from the movable pulley 10 is prevented, improving the stability and reliability.

(s) The cover 14 is vertically slid to allow the access to the inner portion of the mounting platform 5 below the position equal to or less than 120 cm from the floor (F). As a result, it is easier to perform the maintenance work and becomes possible to remove the cable 6 of the probe 2 from the movable pulley 10 and to mount the cable 6 on the movable pulley 10.

(t) Since the movable-pulley lock 15 is installed, it is possible to hold the movable pulley 10 from which the cable 6 has been removed at the position equal to or higher than 60 cm from the floor, thereby making it easier to mount the cable 6 of the probe 2 on the movable pulley 10.

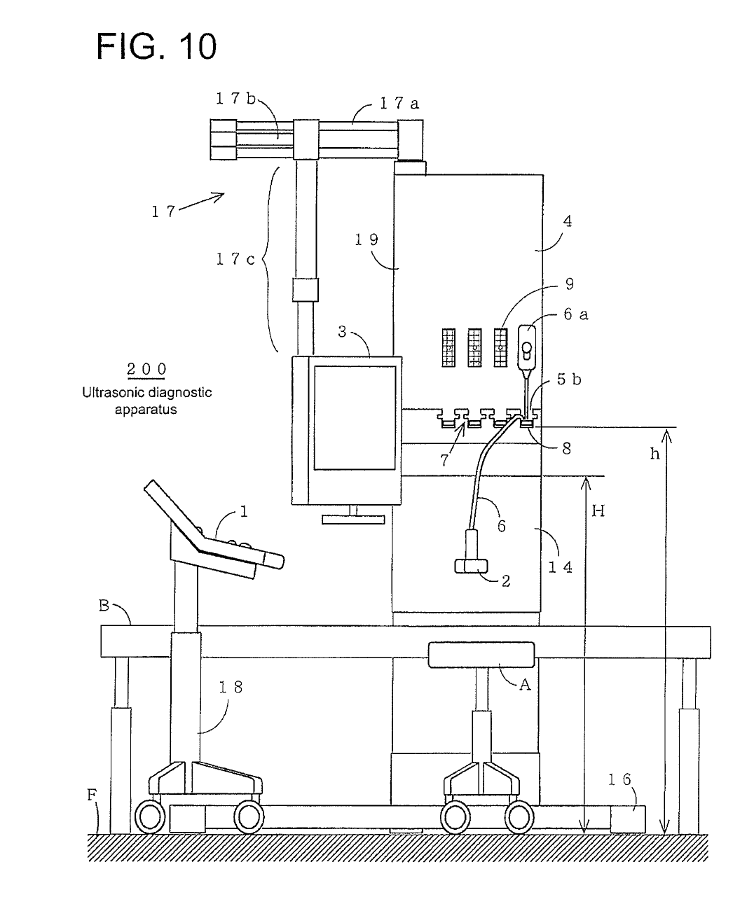

FIG. 10 is a front view showing an ultrasonic diagnostic apparatus 200 according to Embodiment 2.

In the ultrasonic diagnostic apparatus 200, the mounting platform 5 and the column support 19 of Embodiment 1 are integrally formed with the processing device 4.

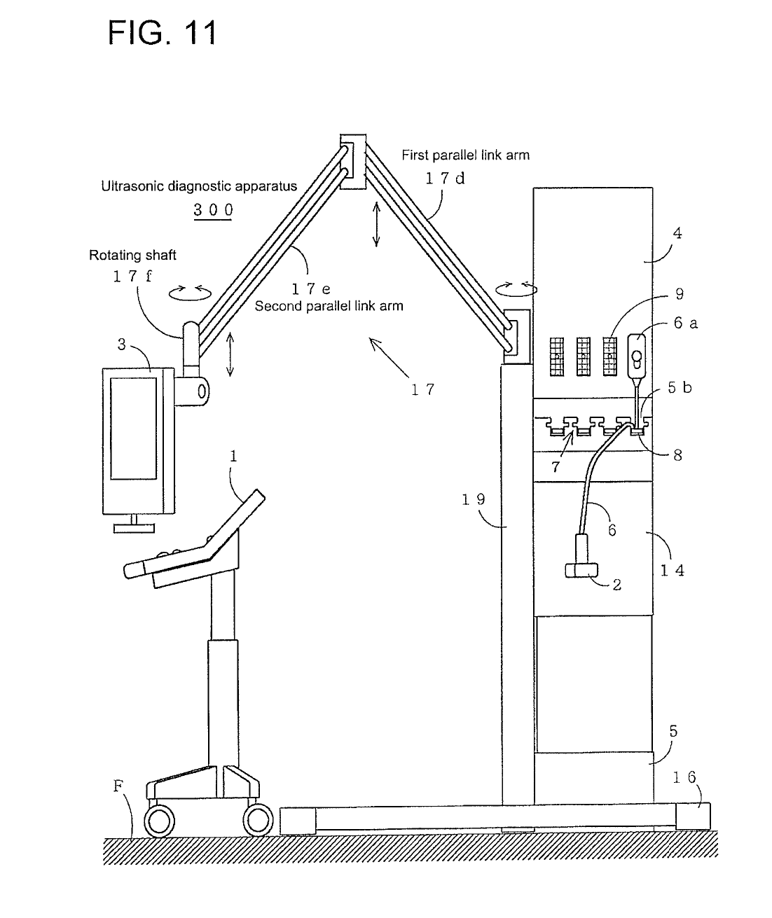

FIG. 11 is a front view showing an ultrasonic diagnostic apparatus 300 of Embodiment 3.

According to the ultrasonic diagnostic apparatus 300, the arm 17 includes: a first parallel link arm 17d extending from the column support 19 in a rotatable and vertically deformable manner; a second parallel link arm 17e extending from the first parallel link arm 17d in a vertically deformable manner; and a rotating shaft 17f extending from the second parallel link arm 17e and rotatably supporting the image display device 3.

The first parallel link arms 17d and 17e make up a parallel link mechanism in which horizontal sides can tilt while vertical sides remain being vertical.

Also, in order to prevent the first parallel link arms 17d and 17e from forming a V shape to lower the position of the joint of the first parallel link arms 17d and 17e to touch the patient, there is provided a stopper (not shown) for preventing the first parallel link arms 17d and 17e from bending in the V shape. That is, the first parallel link arms 17d and 17e can be bent only in a reversed V shape.

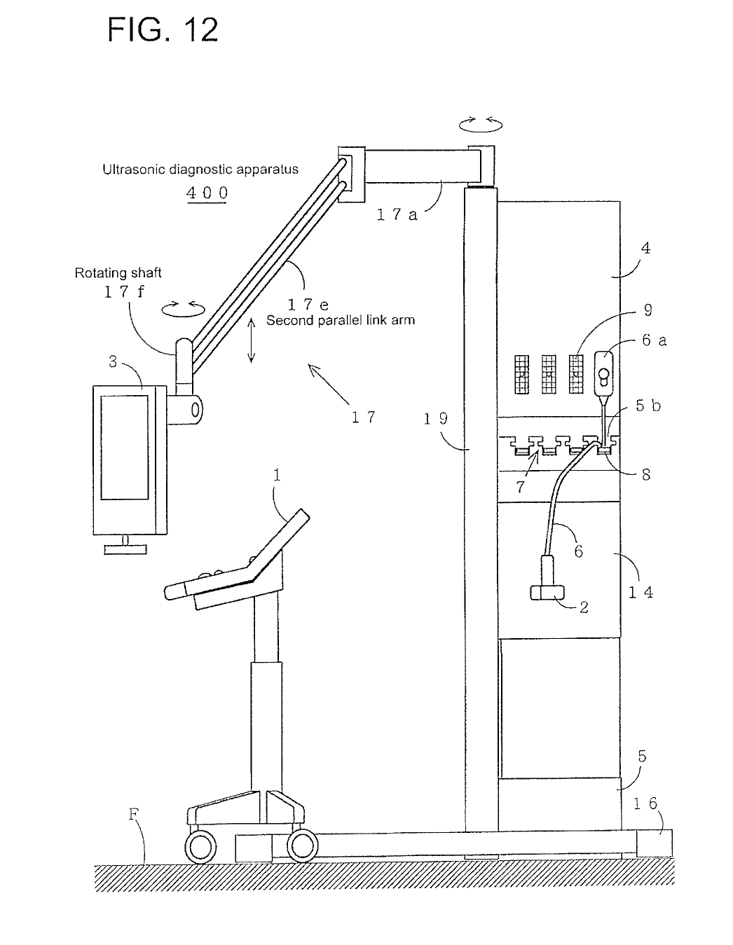

FIG. 12 is a front view showing an ultrasonic diagnostic apparatus 400 of Embodiment 4.

According to the ultrasonic diagnostic apparatus 400, the arm 17 includes: a first horizontal arm 17a extending from the column support 19 in a rotatable manner; a second parallel link arm 17e extending from the first horizontal arm 17a in a vertically deformable manner; and a rotating shaft 17f extending from the second parallel link arm 17e and rotatably supporting the image display device 3.

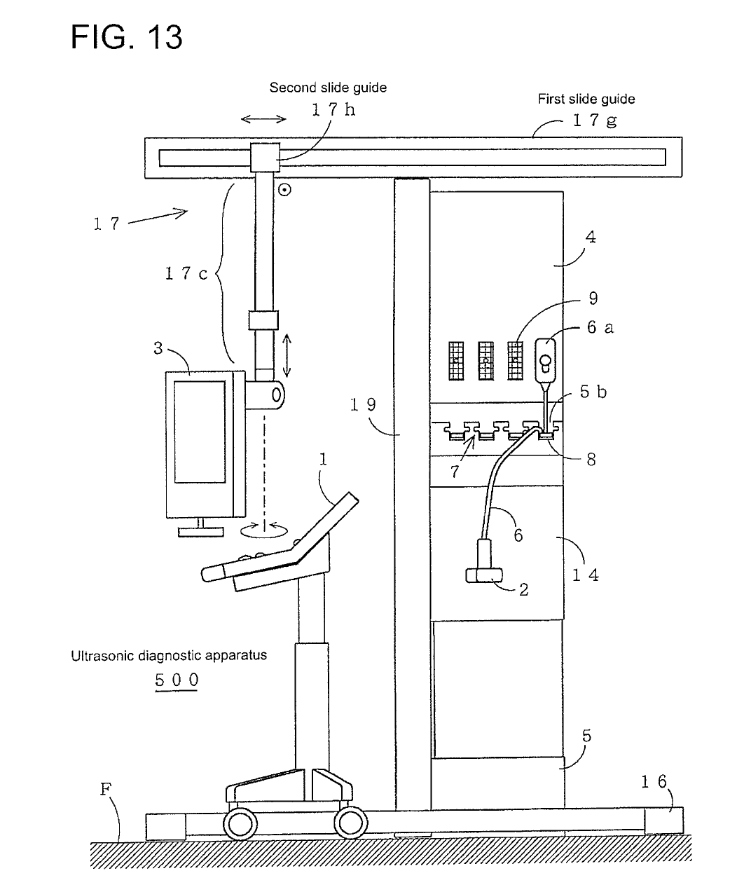

FIG. 13 is a front view showing an ultrasonic diagnostic apparatus 500 of Embodiment 5.

According to the ultrasonic diagnostic apparatus 500, the arm 17 includes: a first slide guide 17g supported on the column support 19 and extending horizontally in a right and left direction; a second slide guide 17h supported slidably along the first slide guide 17g in the right and left direction and extending in a back and forth direction; and an up-and-down arm 17c supported slidably along the second slide guide 17h in the back and forth direction and supporting the image display device 3 in a vertically movable and rotatable manner.

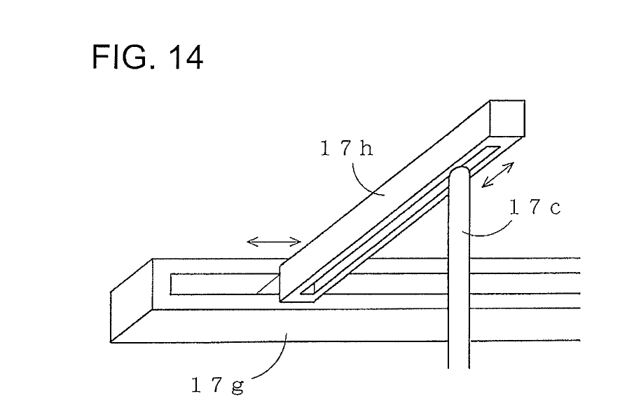

FIG. 14 is an upward view of the second slide guide 17h.

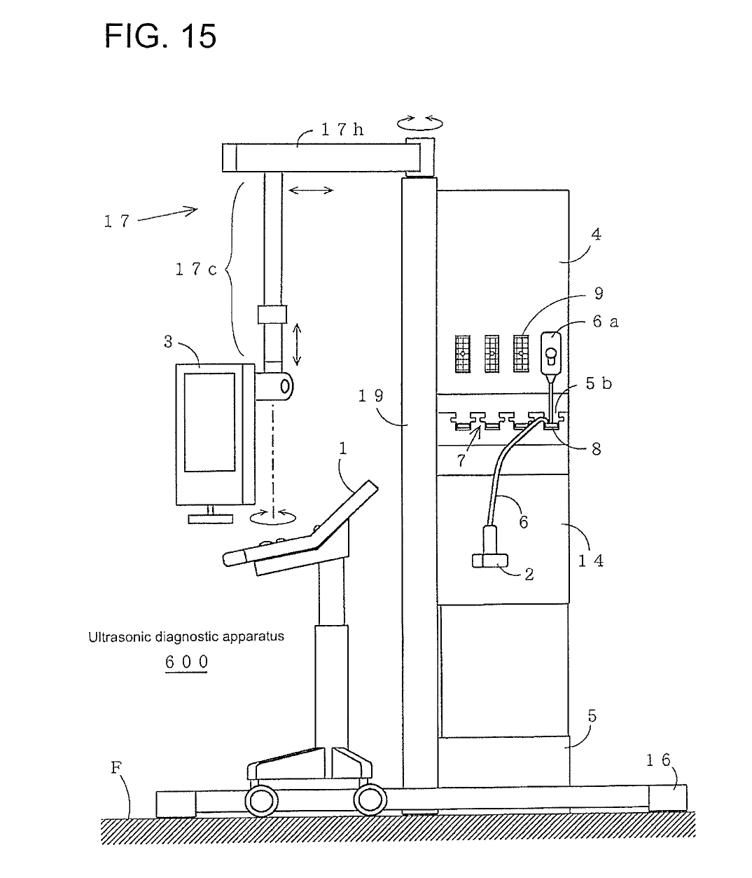

FIG. 15 is a front view showing an ultrasonic diagnostic apparatus 600 of Embodiment 6.

According to the ultrasonic diagnostic apparatus 600, the arm 17 includes: a second slide guide 17h extending from the column support 19 in a rotatable manner; and an up-and-down arm 17c supported slidably along the second slide guide 17h and supporting the image display device 3 in a vertically movable and rotatable manner.

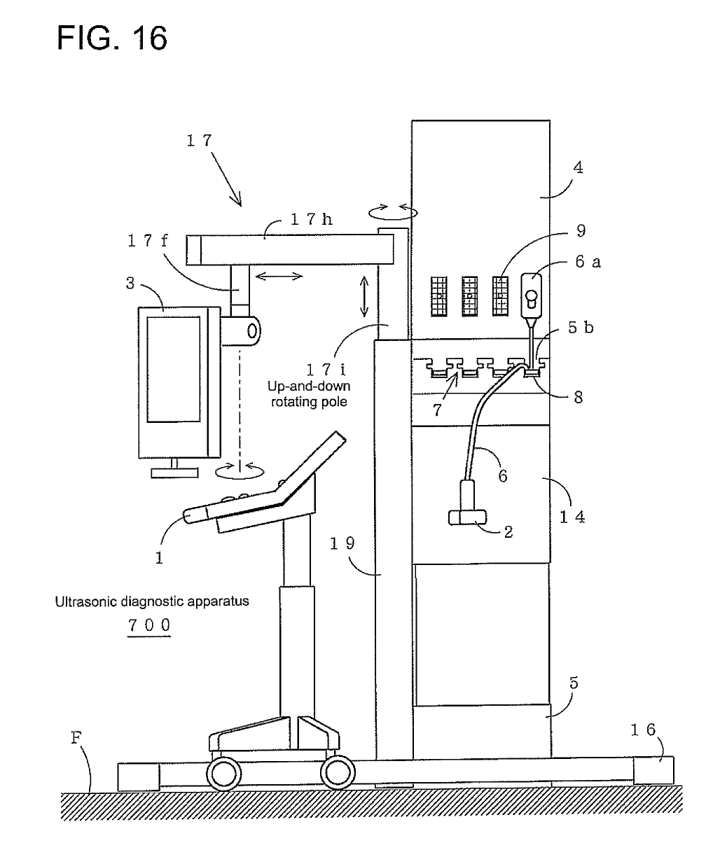

FIG. 16 is a front view showing an ultrasonic diagnostic apparatus 700 of Embodiment 7.

According to the ultrasonic diagnostic apparatus 700, the arm 17 includes: an up-and-down rotating pole 17i installed in the column support 19 in a vertically movable and rotatable manner; a second slide guide 17h extending from the up-and-down rotating pole 17i; and a rotating shaft 17f supported slidably along the second slide guide 17h and rotatably supporting the image display device 3.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus, comprising:
an operating device for an operator to input an instruction;
a probe configured to transmit and receive ultrasonic waves;
an image display device configured to display an ultrasonic image; and
a processing device configured to:
drive said probe based on the input instruction;
generate the ultrasonic image based on a received signal; and
display the ultrasonic image on said image display device, wherein said operating device is formed separately from said processing device and wherein said image display device is supported by one of an arm extending from a column support along said processing device and an arm extending from said processing device.

2. An ultrasonic diagnostic apparatus according to claim 1, wherein said arm comprises: a horizontal arm extending from one of said column support and said processing device; and an up-and-down arm extending from said horizontal arm and supporting said image display device.

3. An ultrasonic diagnostic apparatus according to claim 2, wherein said horizontal arm is configured to rotate at a joint with one of said column support and said processing device.

4. An ultrasonic diagnostic apparatus according to claim 2, wherein said horizontal arm comprises: a first horizontal arm extending from one of said column support and said processing device and a second horizontal arm extending from said first horizontal arm, wherein said second horizontal arm is configured to rotate at a joint with said first horizontal arm.

5. An ultrasonic diagnostic apparatus according to claim 2, wherein a length of said up-and-down arm is variable.

6. An ultrasonic diagnostic apparatus according to claim 2, wherein said up-and-down arm is configured to rotate at a joint with said horizontal arm in a horizontal direction such that the rotation allows said image display device to rotate in a horizontal direction, and wherein said image display device is configured to rotate vertically with respect to said up-and-down arm at a joint with said up-and-down arm.

7. An ultrasonic diagnostic apparatus according to claim 2, wherein said operating device is mounted on a movable platform with a caster.

8. An ultrasonic diagnostic apparatus, comprising:
an operating device for an operator to input an instruction;
a probe configured to transmit and receive ultrasonic waves;
an image display device configured to display an ultrasonic image; and
a processing device configured to:
drive said probe based on the input instruction;
generate the ultrasonic image based on a received signal; and
display the ultrasonic image on said image display device, wherein said operating device is formed separately from said processing device, and wherein said processing device is one of:
mounted on a mounting platform having a portion being equal to or lower than 70 cm from a floor is 10 cm thick or less; and
placed on the floor such that a portion of said processing device is equal to or lower than 70 cm from the floor and is 10 cm thick or less.

9. An ultrasonic diagnostic apparatus according to claim 8, wherein one of a back surface of said processing device and said mounting platform is one of not uneven and a level of unevenness is 1 cm or less.

10. An ultrasonic diagnostic apparatus according to claim 8, wherein one of said processing device said mounting platform comprises a tip-resistant leg.

11. An ultrasonic diagnostic apparatus according to claim 8, wherein said image display device is supported by one of an arm extending from a column support provided along said processing device and an arm extending from said processing device.

12. An ultrasonic diagnostic apparatus according to claim 11, wherein said arm is configured to one of rotate and expand and contract.

13. An ultrasonic diagnostic apparatus according to claim 8, wherein said operating device is mounted on a movable platform with a caster.

14. An ultrasonic diagnostic apparatus according to claim 1, wherein said operating device and said processing device comprise radio communication means for mutual radio communication.

15. An ultrasonic diagnostic apparatus according to claim 1, wherein a cable receiving space is provided inside one of said processing device and said mounting platform for receiving a cable of said probe.

16. An ultrasonic diagnostic apparatus according to claim 15, wherein an opening is provided at a position equal to or higher than 120 cm from one of the floor of said processing device and said mounting platform, and wherein said cable of said probe is received in said cable receiving space through said opening.

17. An ultrasonic diagnostic apparatus according to claim 15, wherein a cable stopper is provided for preventing said cable from coming into and going out of said cable receiving space.

18. An ultrasonic diagnostic apparatus according to claim 17, wherein said cable stopper comprises a slit that is narrower than an outside diameter of said cable.

19. An ultrasonic diagnostic apparatus according to claim 16, wherein a roller which can be in contact with an undersurface of said cable is provided in said opening.

20. An ultrasonic diagnostic apparatus according to claim 16, wherein a probe connector is provided above said opening of said processing device.
